(19) United States
(12) Patent Application Publication
(10) Pub. No.: US 2018/0330823 A1
(43) Pub. Date: Nov. 15, 2018

(54) METHOD AND SYSTEM FOR GENERATING ESTIMATION DATA FOR POTENTIAL INJURY STATES

(71) Applicant: Siemens Healthcare GmbH, Erlangen (DE)

(72) Inventors: Philipp HOELZER, Baltimore, MD (US); Razvan Ionasec, Nuernberg (DE); Sebastian Schmidt, Weisendorf (DE); Amitkumar Bhupendrakumar Shah, Nuernberg (DE)

(73) Assignee: Siemens Healthcare GmbH, Erlangen (DE)

(21) Appl. No.: 15/974,883

(22) Filed: May 9, 2018

(30) Foreign Application Priority Data
May 15, 2017 (EP) 17171122.9

(51) Int. Cl. G16H 50/20; G07C 5/00; G16H 10/60; G16H 50/70

(52) U.S. Cl. CPC G16H 50/20; G16H 50/70; G16H 10/60; G07C 5/008

(57) ABSTRACT

A method is for automatically generating estimation data for potential injury states of at least one person following an incident involving physical force or violence. In an embodiment, the method includes providing personal data relating to the person; providing incident data relating to the incident involving physical force or violence; and determining the estimation data for likely injury states via a modeling process based on the personal data and the incident data. At least one embodiment of the invention further relates to a respective system and vehicle compatible therewith.

### PRIORITY STATEMENT

The present application hereby claims priority under 35 U.S.C. § 119 to German patent application number EP 17171122.9 filed May 15, 2017, the entire contents of which are hereby incorporated herein by reference.

### FIELD

At least one embodiment of the invention relates to a method and a system for automatically generating estimation data for potential injury states of at least one person following an incident involving physical force or violence.

### BACKGROUND

Following an accident, an act of violence or some other event involving physical force or violence acting on a person or a group of persons, it is important that injured persons receive the help required as a result of the respective injuries as quickly as possible.

Normally, emergency services are notified for this purpose by an emergency call from a person involved or a passer-by.

A disadvantage of this course of action is that valuable time is lost unnecessarily between the incident involving force or violence and the emergency call. Furthermore, the situation may occur whereby an accident or an act of violence takes place at an isolated location with no witnesses and the persons involved are unable to make an emergency call. Thus, for instance, it occasionally happens that accident victims whose vehicle has left a road are not discovered until hours or even days after the crash.

It is for this reason that in the future the European Union plans to roll out a system called “eCall”, which reports a vehicle accident automatically.

### SUMMARY

The inventors have discovered that the aforementioned system has a disadvantage in that even though an accident is reported, no further important data is sent relating to the possible injuries or the number of persons involved. As a result, valuable time may once again elapse unnecessarily before the necessary resources for evacuating all of the injured, for diagnosis or for treatment at the scene are selected.

At least one embodiment of the present invention provides an improved method and a correspondingly improved system by which at least one of the above-described disadvantages are reduced or avoided.

At least one embodiment of the present invention provides for a method and at least one embodiment of the present invention provides for a system. A vehicle, provided in at least one embodiment, advantageously interacts cooperatively with such a system or such a method.

The method according to at least one embodiment of the invention comprises:

a) Providing personal data relating to the person. The personal data (also known as “personally identifiable information”) is data which provides information about the physical and/or, where appropriate, also the psychological state or frame of mind of the person(s) involved in the incident involving force or violence prior to the incident involving force or violence or, as the case may be, without any occurrence of the incident.

b) Providing incident data relating to the incident involving physical force or violence. The incident data preferably comprises measured values and is data relating to injury-relevant physical effects on the person(s) involved in the incident involving force or violence.

c) Determining estimation data for likely injury states via a modeling process based on the personal data and the incident data. Preferably, the injury data also comprises information relating to the probable severity of the injury states. The modeling process preferably comprises a simulation or a routine for utilizing direct functional interrelationships, in particular based on tables. The results are preferably probability values for the presence of possible injuries and preferably also the severity thereof.

The system of at least one embodiment comprises at least:

a) an interface for receiving personal data,

b) an interface for receiving incident data relating to the incident involving physical force or violence, and

c) a modeling unit which is configured to determine estimation data for likely injury states of possible injuries by way of a modeling process based on the personal data and the incident data.

In that respect, at least one embodiment of the invention is directed to a computer program product having a computer program which can be loaded directly into a memory device of a control device of a computed tomography system and has program sections for performing all steps of the method according to at least one embodiment of the invention when the program is executed in the control device. As well as the computer program, such a computer program product may, where appropriate, comprise additional constituent parts such as e.g. documentation and/or additional components, including hardware components, such as e.g. hardware keys (dongles, etc.) to allow use of the software.

Also preferred is a non-transitory computer-readable medium on which program sections that can be read in and executed by a computer unit are stored for the purpose of performing all steps of the method according to the invention when the program sections are executed by the computer unit.

Further particularly advantageous embodiments and developments of the invention will become apparent from the dependent claims, as well as from the following description. In this regard, the claims of one claims category may also be developed analogously to the dependent claims or parts of the description of a different claims category, and in particular also individual features of different example embodiments or variants may be combined to create new example embodiments or variants.

### BRIEF DESCRIPTION OF THE DRAWINGS

The invention is explained once again in more detail below with reference to the attached figures and with the aid of example embodiments. Like components in the various figures are labeled with identical reference numerals. The figures are generally not to scale. In the figures:

FIG. 1 shows a schematic representation of a preferred embodiment variant of a method according to the invention,

FIG. 2 shows a schematic representation of a preferred embodiment variant of a system according to the invention,

FIG. 3 shows a schematic intended to illustrate possible types of personal data,

FIG. 4 shows a schematic intended to illustrate possible types of incident data and the provision of the data,

### DETAILED DESCRIPTION OF THE EXAMPLE EMBODIMENTS

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

Various example embodiments will now be described more fully with reference to the accompanying drawings in which only some example embodiments are shown. Specific structural and functional details disclosed herein are merely representative for purposes of describing example embodiments. Example embodiments, however, may be embodied in various different forms, and should not be construed as being limited to only the illustrated embodiments. Rather, the illustrated embodiments are provided as examples so that this disclosure will be thorough and complete, and will fully convey the concepts of this disclosure to those skilled in the art. Accordingly, known processes, elements, and techniques, may not be described with respect to some example embodiments. Unless otherwise noted, like reference characters denote like elements throughout the attached drawings and written description, and thus descriptions will not be repeated. The present invention, however, may be embodied in many alternate forms and should not be construed as limited to only the example embodiments set forth herein.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “exemplary” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Before discussing example embodiments in more detail, it is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments of the present invention. This invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one embodiment of the invention relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

The method according to at least one embodiment of the invention provides an automatic generation of estimation data concerning potential injury states of at least one person following an incident involving physical force or violence.

The estimation data concerning potential injury states, referred to in the following in the interests of easier readability also as “injury data”, in this context comprises assessments or estimates for which injuries might have occurred. Preferably, it comprises probability values or “probability data” for the presence in a person of an injury state known to the method. The estimation data, in particular probability values, may also be formed for two or more different possible injuries. For example, the injury data may include the information that there is a 30% probability of a leg fracture, a 50% probability of a knee injury and a 90% probability of a sprained ankle being present. The injury data thus delivers an estimation of the state of health of at least one person. It is, however, also possible that the estimation data or injury data does not relate to any quantitative indications concerning probabilities, but points qualitatively to potential injuries. For example, the estimation data may contain the qualitative indication “suspected basal skull fracture” or similar.

The incident involving physical force or violence, or the incident involving force or violence “subject to the laws of physics”, since physical forces are in play, is an incident in which potentially a person may potentially have come to harm due to force or violence. This may be an accident or an act of violence, for example.

The method according to at least one embodiment of the invention comprises:

a) Providing personal data relating to the person. The personal data (also known as “personally identifiable information”) is data which provides information about the physical and/or, where appropriate, also the psychological state or frame of mind of the person(s) involved in the incident involving force or violence prior to the incident involving force or violence or, as the case may be, without any occurrence of the incident.

b) Providing incident data relating to the incident involving physical force or violence. The incident data preferably comprises measured values and is data relating to injury-relevant physical effects on the person(s) involved in the incident involving force or violence.

c) Determining estimation data for likely injury states via a modeling process based on the personal data and the incident data. Preferably, the injury data also comprises information relating to the probable severity of the injury states. The modeling process preferably comprises a simulation or a routine for utilizing direct functional interrelationships, in particular based on tables. The results are preferably probability values for the presence of possible injuries and preferably also the severity thereof.

The modeling process may in this case comprise a modeling of the entire incident involving force or violence or of the course of events, or of only a part of the incident involving force or violence which is relevant to the effects of the incident involving force or violence on the persons involved, or a modeling of the effects of the incident involving force or violence on the persons involved.

A modeling process may in this case utilize e.g. the incident data for estimating the interactions of as many objects and persons as possible during the incident involving force or violence, e.g., in the case of an accident, of the forces which have acted on the occupants of a vehicle, preferably on the individual parts of the body and organs of the occupants. The personal data is preferably used to estimate the effects which the cited interactions could have had on the physical constitution of the respective persons. Thus, the probability of bone fractures is generally greater in the case of older persons than in the case of younger persons.

The system according to at least one embodiment of the invention serves for automatically generating estimation data for potential injury states of at least one person following an incident involving physical force or violence. It uses in particular the above-described method according to at least one embodiment of the invention.

The system of at least one embodiment comprises at least:
a) an interface for receiving personal data,
b) an interface for receiving incident data relating to the incident involving physical force or violence, and
c) a modeling unit which is configured to determine estimation data for likely injury states of possible injuries by way of a modeling process based on the personal data and the incident data.

A vehicle according to at least one embodiment of the invention is equipped with a sensor system for recording incident data. It is characterized in that it is equipped with an interface which is configured for communication with an interface for receiving incident data of a system according to at least one embodiment of the invention. In addition, the vehicle is preferably equipped also with a database containing personal data pertaining to persons normally making use of the vehicle and additionally comprises an interface which is configured for communication with an interface for receiving incident data of a system according to at least one embodiment of the invention. This has the advantage that relevant incident data and, where applicable, also personal data can very quickly be sent to the system according to at least one embodiment of the invention by the most direct route or is very quickly available to the method according to at least one embodiment of the invention.

The estimation data for likely injury states can be provided automatically to the relevant emergency services via known channels, preferably via data links or by wireless transfer, e.g. via the mobile communications network. In this case the system may be located together with the transmission unit for example in an object involved, e.g. in a vehicle, or be available as an application in a mobile communications device, but may also be present at another location, e.g. a central point, which basically is immaterial as long as it receives the necessary data. The transmission unit may be part of the system, but may also be an independent transmission unit.

Most of the aforementioned components of the system may be realized wholly or in part in the form of software modules in a processor of a corresponding control device. A largely software-based implementation has the advantage that infrastructure already in use previously in the prior art can also be easily upgraded by way of a software update in order to operate in the manner according to at least one embodiment of the invention.

In that respect, at least one embodiment of the invention is directed to a computer program product having a computer program which can be loaded directly into a memory device of a control device of a computed tomography system and has program sections for performing all steps of the method according to at least one embodiment of the invention when the program is executed in the control device. As well as the computer program, such a computer program product may, where appropriate, comprise additional constituent parts such as e.g. documentation and/or additional components, including hardware components, such as e.g. hardware keys (dongles, etc.) to allow use of the software.

Also preferred is a non-transitory computer-readable medium on which program sections that can be read in and executed by a computer unit are stored for the purpose of performing all steps of the method according to the invention when the program sections are executed by the computer unit.

Further particularly advantageous embodiments and developments of the invention will become apparent from the claims, as well as from the following description. In this regard, the claims of one claims category may also be developed analogously to the dependent claims or parts of the description of a different claims category, and in particular also individual features of different example embodiments or variants may be combined to create new example embodiments or variants.

Furthermore, vehicles may also contain individual components of the system according to at least one embodiment of the invention or a complete system and consequently autonomously supply the predetermined information following an accident, which constitutes a great advantage in particular in the case of accidents occurring at isolated locations.

Preferably, the personal data includes biometric data, data relating to identity and/or to physical constitution, in particular to known preexisting conditions. Preferred personal data is data belonging to the group composed of age, gender, size, weight, preexisting conditions, and data from medical records. Important pointers to the physical constitution of a person may already be derived from the data.

Preferably, the incident data includes data relating to one or more of the following cases. This may for example be purely numeric data in each case or data of a semantic data model. The format of the data should be chosen such that, in the modeling step, the data can be processed or placed into a relevant context with respect to the result.

i) Incident description: This is data relating to the type of incident. For example, whether it concerns a car accident, a motorbike accident, an accident involving pedestrians, a stabbing, an exchange of gunfire, a blunt force trauma, a fall, or other possible incidents involving force or violence.

ii) Speed, direction of movement, relative positions, accelerations/forces acting on the person or other physical circumstances in connection with the incident involving force or violence. In this case the relevant data pertaining to the persons involved can provide important clues to potential injuries, but also relevant data pertaining to objects, e.g. vehicles or data pertaining to objects that could have caused injury to persons (e.g. bullets from a handgun).

iii) Relative orientation of the person(s) and/or of objects involved. This data can advantageously be used to estimate which objects could have interacted with which persons.

iv) Description of objects involved. The latter can provide important pointers to the type of potential injuries, e.g. whether the injury could have been caused by a knife, a bullet or a blunt object.

v) Partial accelerations of the parts of the body of the person or penetration depths of objects into the person. This data is helpful in estimating which regions of the body might exhibit which injuries.

vi) Physical effect of a plurality of persons subjected to the incident involving force or violence on at least one of the persons involved. This data is helpful in deriving injury patterns which could have resulted due to the interaction of parts of the body of different persons on one another, e.g. punches, but also a possible penetration of organs of one person through a compound fracture of another person.

Preferably, incident data, in particular data relating to a vehicle accident, comprises data belonging to the group composed of vehicle type (weight category, shape), speed, impact angle, orientation of a vehicle with respect to the victim, characteristics of the hood or the vehicle body (smooth or projecting objects such as e.g. a hood ornament or a bullbar), deformations of the vehicle at the point of impact, and the point of impact on the person (hip, chest, leg, etc.).

Preferably, the incident data is acquired or obtained from sensors or persons. One possibility in this case is that the incident data comprises values measured by sensors which are coupled to at least one object involved in the incident involving physical force or violence. Such sensors are e.g. sensors in a vehicle or in a mobile communications device carried by a person, or in a smartwatch or fitness sensors. However, the incident data may also include data recorded by at least one recording unit capturing the incident involving physical force or violence, e.g. a traffic camera, or include witness statements or video recordings taken by witnesses. For example, it is possible to establish the circumstances surrounding the act of violence or accident from video footage of the incident involving force or violence, and also to estimate speeds. This applies similarly to witness statements, which can be analyzed via speech recognition systems.

Other types of sensors are also preferred, in particular those belonging to the group composed of sensors for monitoring human beings, e.g. devices for measuring the oxygen saturation in the blood or for recording an ECG or EEG, sensors for measuring acceleration or other mechanical variables, sensors for measuring temperature, sensors for measuring noises or voices, e.g. microphones, or sensors which are able to detect the deployment of an airbag, e.g. sensors in the triggering electronics of a vehicle airbag.

Preferably, estimation data for potential injury states of a plurality of persons is generated, in which case in particular the interaction of the persons involved in the incident involving physical force or violence is taken into account in the modeling process. In this way it is possible to model injury patterns that among other things are caused by other persons involved. In this case the modeling can be performed sequentially starting from each of the persons involved or simultaneously starting from some or all of the persons. This could be accomplished in particular by solving a cost function that is to be minimized.

In this case the modeling process preferably comprises calculations which relate to the interaction of the persons with regard to the estimation data for likely injury states of the relevant persons and/or of the remaining persons.

The generation of estimation data for potential injury states with the aid of the modeling preferably comprises a simulation and/or a dictionary-based search or, as the case may be, a selection of entries from a dictionary according to the available personal data and incident data. Calculations based on finite elements, differences or volumes, Monte Carlo simulations or Markow chains are preferred in this context. Also preferred are graphical and/or volume-based simulations by way of persons or objects modeled as solid bodies and via a simulation of physical principles based on incident data. A modeling process based on machine learning principles is also preferred. In particular, an algorithm is initially trained in this regard using sets of data comprising incident data and personal data. The trained algorithm then delivers injury data as a function of its training status on the basis of the current personal data and incident data in each case. Generally, the algorithm is inherently intended to represent the relationship between situation and result.

A preferred modeling process is explained in greater detail below taking a car accident as an example.

a) First, information is input concerning the circumstances surrounding the accident and the persons involved. The incident data and the personal data are used for this purpose.

Such data may be, for example, data relating to the vehicle type, the speed, the angle of impact, the orientation with respect to the victim, deformations of the vehicle, and the point of impact on the person.

b) Next, a database is provided containing data relating to simulations that have been carried out previously based on available information relating to other incidents involving force or violence. These simulations in this case comprise both the circumstances surrounding the accident or the scenario of the incident involving force or violence and the medical impact on the persons involved in the respective other incidents involving force or violence.

c) The information (incident data and personal data) relating to the current incident involving force or violence is synchronized with entries from the aforethe database in order to identify a similar accident situation and to perform corresponding simulations as in the case of the similar accident or to obtain the corresponding data from the database.

d) This enables a prediction of the medical effects on casualties to be made based on the data of the database or the simulation specified by the database.

A preferred database containing simulations of a collision with an object comprises simulated data of a collision from many different (where applicable, all) possible spatial directions, a collision with many different (where applicable, all) possible shapes, and a collision with many different (where applicable, all) possible materials.

Simulations are preferably based on information of the group composed of
typical mechanical properties of organs, such as e.g. deformations, bone fractures, vascular ruptures,
mechanical properties of materials, such as e.g. splinters, edge formation, strength, that are used in or on objects involved, e.g. automobiles,
position of organs in a person's body,
chemical compatibility of human tissue with materials involved in the incident involving force or violence, and
biomechanics of the body, such as e.g. the spinal column, in order to estimate whether a whiplash injury could possibly be present following an incident involving force or violence.

Suitable boundary conditions are based on the conservation of energy and the conservation of momentum, as well as on elastic properties of materials that may serve to attenuate accelerations, and on coefficients of friction.

Suitable examination and/or treatment methods for the targeted examination and/or treatment of the person are preferably identified based on the determined estimation data for potential injury states and, where applicable, the probable severity thereof. It is preferred in this context to identify the group composed of prioritization of the likely necessary examinations, assessment of the seriousness of the injuries, and recommendation for necessary interventions.

A protocol for controlling a medical apparatus is preferably generated from the injury data, e.g. for the purpose of controlling a computed tomography (CT) system, a magnetic resonance tomography (MRT) system, a positron emission tomography (PET) system, a single-photon emission computed tomography (SPECT) apparatus, an anatomical fluoroscopy apparatus, an ultrasound imaging apparatus, an optical coherence tomography (OCT) apparatus, photoacoustic apparatus, or an ultra-wideband imaging (UWB imaging) apparatus. This enables a faster subsequent diagnosis by a physician, since there is no need firstly to produce the control data for the apparatus. An already generated control data set can also be adapted by a physician to match new findings in a simple and time-saving manner based on the physician's specialized knowledge.

For this purpose, the system preferably comprises a determination unit which automatically determines capacities of emergency and diagnostic equipment based on the identified possible injuries and, where applicable, the probable severity thereof, as well as preferably based on identified suitable examination methods.

Probably necessary capacities of emergency and diagnostic equipment are preferably determined based on the determined estimation data for potential injury states and, where applicable, the probable severity thereof, as well as preferably based on identified suitable examination methods, and are preferably reserved and in particular also notified. This has the advantage of facilitating a speedy allocation of casualties to free emergency and examination capacities, and as a result enables a prompt treatment to be carried out.

For this purpose, the system preferably comprises a notification unit which automatically reserves and/or notifies capacities of emergency and diagnostic equipment based on the determined possible injuries and, where applicable, the probable severity thereof, as well as in particular based on identified suitable examination methods. This notification can be transmitted for example by way of the known channels cited hereinbelow. In particular, an eCall system can be improved by way of at least one embodiment of the present invention and use can be made of its communication channels.

Within the scope of the notification, the determined estimation data for potential injury states can preferably be transmitted together with information relating to the objects or persons involved, in particular to the expected casualty figures.

The interface for receiving incident data of the system is preferably configured for accessing a computing system, preferably via the Internet of Things (IoT), a computer cloud, wireless networks, e.g. 4G, 5G, or internet protocols, e.g. IPv6. This enables the required incident data to be obtained in a quick and uncomplicated manner.

The interface for receiving personal data is preferably configured for accessing storage media, preferably databases, comprising electronic patient data, preferably conforming to HL7 standards, or medical image transfer systems, preferably conforming to DICOM standards (DICOM=Digital Imaging and Communications in Medicine).

FIG. 1 illustrates a preferred method via a block diagram.

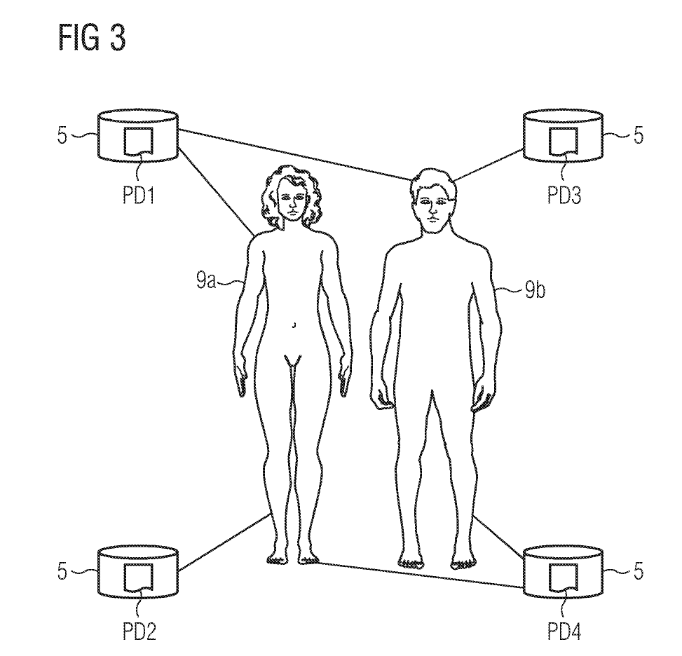

Block I shows the provision of personal data PD for the method. As indicated in FIG. 3, the personal data PD may comprise different personal data PD1, PD2, PD3 or PD4.

FIG. 3 shows a schematic view of the allocation of different personal data PD1, PD2, PD3 and PD4 pertaining to two persons 9a and 9b to different databases 5. As can be seen in the databases 5 at top left and bottom right, personal data PD1, PD4 pertaining to both persons may be present in the same databases, e.g. when the persons are bound by family ties and/or are undergoing treatment from the same physician, though the sets of data may also be present independently of one another in different databases 5.

For example, the data PD1 is data pertaining to the persons' identity, e.g. name, address, age. This can by all means be present in one file of one database in the case of people connected by family ties. Equally, the personal data PD4 for both persons may reside in a common database, the data comprising for example biometric data such as e.g. size or weight. The data PD2 and PD3 are for example medical data pertaining to the respective persons, the data residing in separate databases because the persons in this example are undergoing treatment from different physicians.

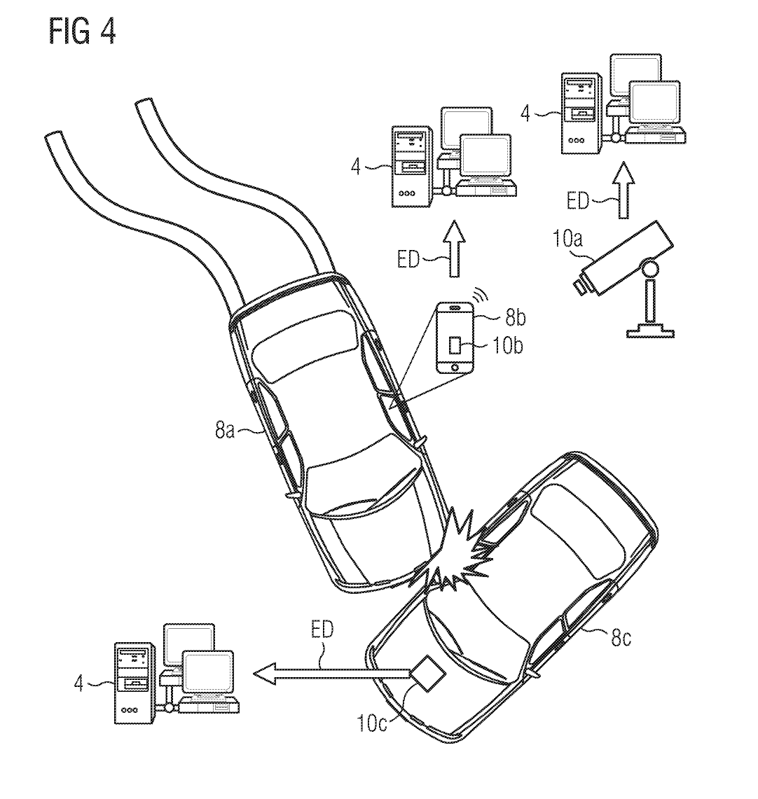

Block II shows the provision of incident data ED for the method. As is indicated in FIG. 4, which will be explained in greater detail later, the incident data ED may be obtained from different sensors 10a, 10b, 10c. Generally, the data is recorded by a body endowed with authority. The bodies endowed with authority include e.g. mobile communications providers, traffic monitoring agencies, vehicle management organizations. In most cases the data is stored in computing systems 4 which provide the data, on request where applicable. Thus, the different incident data ED accessed by the method may originate from a plurality of different computing systems 4 or be provided by the same.

Block III illustrates the editing of the data into a format which permits a modeling process M. This step is not absolutely necessary, at least when the data ED or PD is already present in a suitable format or the modeling process is configured for processing the respective formats.

Block IV symbolizes the modeling step. In this case the modeling M takes place based on the personal data PD and the incident data ED, e.g. a simulation of the circumstances surrounding the accident. The injury data VD is produced using the modeling process.

The modeling process uses the incident data in order to estimate the interactions of persons and objects during the incident involving force or violence. For example, forces in an accident that have acted on the occupants of a vehicle, partially on regions of the body where applicable, are extrapolated from speeds or accelerations. In order to estimate the injuries potentially resulting therefrom, that personal data is used which may provide indications as to the effect that forces acting on a person could have on the body of that person.

The injury data VD is provided in block V. The data can be used e.g. for identifying examination and/or treatment methods for the targeted examination and/or treatment of the person (9a, 9b) or for producing a protocol SP for controlling a medical apparatus, such as e.g. a tomography system 11.

Block VI represents the determination and reservation of probably necessary capacities of emergency equipment R and/or examination equipment based on the injury data VD. For example, emergency services are notified according to the number of determined potential casualties and the likely injuries and, if necessary, beds are reserved in hospitals in advance. It would, however, also be possible to send e.g. a control protocol for a computed tomography system, which permits a prompt examination of an injured person to be conducted immediately after the latter's admission into the appropriate institution.

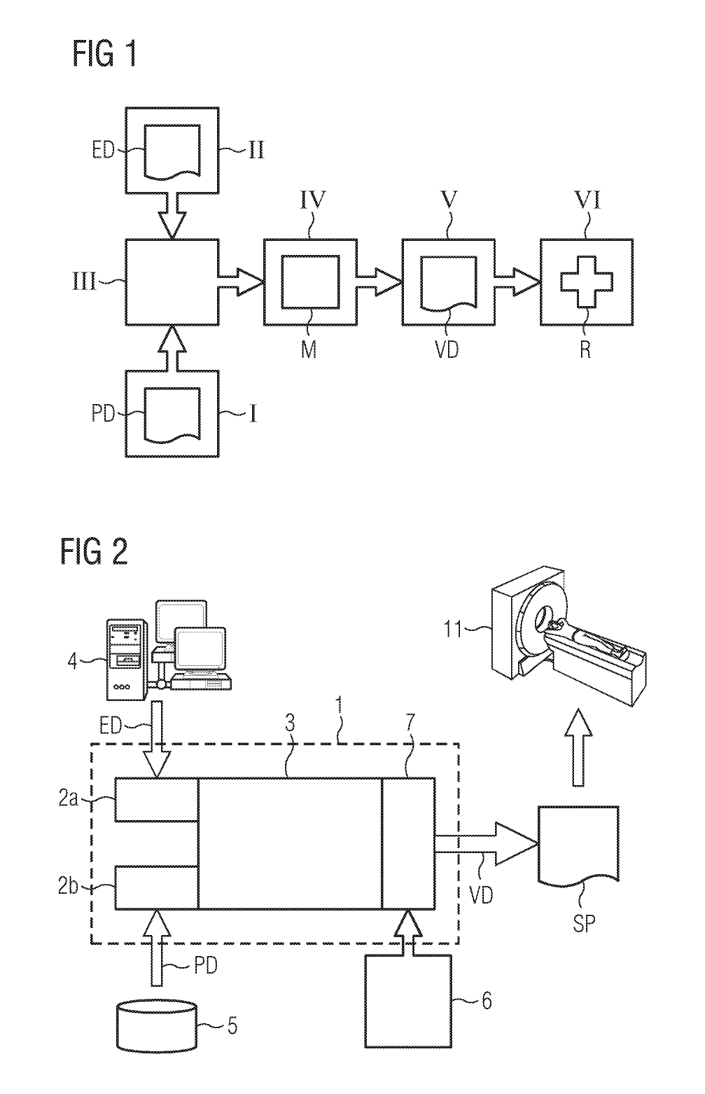

FIG. 2 shows a schematic representation of a preferred system 1. In this case the personal data PD is stored in a database 5 which, as has already been mentioned above with reference to FIG. 3, may also symbolize a group of different databases 5. The incident data ED is stored in a computing system 4 which, as has already been mentioned above with reference to FIG. 4, may also symbolize a group of different computing systems 4.

The system 1 is provided with the personal data PD by the database 5 via the interface 4b and with the incident data ED via the computing system 4 via the interface 4a. The data may be reformatted or edited if necessary, as has been mentioned already with reference to the method, though this is not explicitly depicted in addition in this case.

The injury data VD is generated in the modeling unit 3 by a modeling M of the effects of the incident involving force or violence on the persons involved. The injury data VD can then be made available to services via a notification unit 7, as has already been described in the course of the method. This can happen based on data determined by a determination unit 6. For example, the determination unit 6 can notify the system of which capacities are available or free in which hospital.

The provision of injury data VD for generating a control protocol SP for a tomography system 11 is illustrated here. In this case the control protocol SP may be generated externally, though it may also be generated directly via the system 1.

FIG. 4 illustrates an incident involving force or violence in the form of a car accident. In the accident, one vehicle 8a has crashed into the side of another vehicle 8c. A mobile communications device 8b is disposed in the vehicle 8a, which mobile communications device 8b could for example have been carried by the driver and could possibly have also caused injuries. The vehicles involved in the accident and the mobile communications device may also be referred to as “objects” within the meaning of at least one embodiment of the invention.

An acceleration sensor 10b is disposed in the mobile communications device 8b, which acceleration sensor 10b has sent data to a computing system 4 of the mobile communications provider. The data also includes incident data ED that has been recorded during the accident and could provide information about the acceleration of the driver during the accident.

Also disposed in the vehicle 8c is a vehicle sensor 10c, which measures the respective state of the vehicle 8c, including, among other variables, accelerations acting on the vehicle 8c. The vehicle sensor 10c sends the data to a computing system 4 of the vehicle manufacturer. This likewise includes incident data ED that has been recorded during the accident and could provide information about the speed and acceleration of the accident vehicle 8c during the accident.

Thirdly, the accident has been recorded by a traffic camera 10a, the data of which, including, inter alia, the incident data ED, has been sent to a computing system 4 of the traffic monitoring agency.

All the incident data ED of the different computing systems 4 can be used by at least one embodiment of the inventive method or at least one embodiment of the inventive system for modeling M the effects of the incident involving force or violence on the persons involved. The more data that is available to the inventive system 1 or method, the more accurate will be the result.

In conclusion, it is pointed out once again that the methods described in detail in the foregoing and the illustrated system 1 are simply example embodiments which can be modified in a wide variety of ways by the person skilled in the art without leaving the scope of protection of the invention. Furthermore, the use of the indefinite articles “a” or “an” does not exclude the possibility that the features in question may also be present more than once. Similarly, the terms “unit” and “module” do not rule out the possibility that the components in question consist of a plurality of cooperating subcomponents, which, where necessary, may also be spatially distributed.

The patent claims of the application are formulation proposals without prejudice for obtaining more extensive patent protection. The applicant reserves the right to claim even further combinations of features previously disclosed only in the description and/or drawings.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

None of the elements recited in the claims are intended to be a means-plus-function element within the meaning of 35 U.S.C. § 112(f) unless an element is expressly recited using the phrase “means for” or, in the case of a method claim, using the phrases “operation for” or “step for.”

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for automatically generating estimation data for potential injury states of at least one person following an incident involving physical force or violence, comprising:
providing personal data relating to the at least one person;
providing incident data relating to the incident involving physical force or violence; and
automatically generating the estimation data for potential injury states via a modeling process based on the personal data and the incident data.

2. The method of claim 1, wherein the personal data includes data belonging to a group including at least one of age, gender, size, weight, preexisting conditions, and data from medical records.

3. The method of claim 1, wherein the incident data includes data belonging to a group including at least one of
incident description
speed,
direction of movement,
relative positions,
relative orientation of at least one of a person or of persons and objects involved,
description of objects involved,
at least one of forces and accelerations acting on persons,
partial accelerations of parts of the body of persons,
depth of penetration of objects into persons, and
physical effect of a plurality of persons subjected to the incident involving force or violence on at least one of the persons.

4. The method of claim 1, wherein the incident data is obtained and provided via at least one of
values measured by sensors coupled to at least one object involved in the incident involving physical force or violence,
data recorded by at least one recording unit recording the incident involving physical force or violence, and
witness statements concerning the incident involving physical force or violence.

5. The method as claimed of claim 1, wherein
the automatically generating of the estimation data for potential injury states of at least one person includes automatically generating the estimation data for a plurality of persons.

6. The method of claim 1, wherein the automatically generating of the estimation data for potential injury states via the modeling process includes at least one of a simulation and a dictionary-based search.

7. The method of claim 1, wherein at least one of
at least one of suitable examination and treatment methods for targeted at least one of examination and treatment of the person are identified based on the determined estimation data for potential injury states,
at least one of a prioritization of the likely necessary examinations, an assessment of the seriousness of the injuries and a recommendation for necessary interventions is performed, and
a protocol for controlling a medical apparatus is produced.

8. The method of claim 1, wherein probably necessary capacities of at least one of emergency and examination equipment is determined and reserved based on the determined estimation data for potential injury states.

9. A system for automatically generating estimation data for potential injury states of at least one person following an incident involving physical force or violence, comprising:
an interface to receive personal data;
an interface to receive incident data relating to the incident involving physical force or violence;
a modeling unit, configured to automatically generate estimation data for potential states of possible injuries via a modeling process based on the personal data and the incident data.

10. The system of claim 9, wherein the interface to receive incident data is configured to access a computing system.

11. The system of claim 9, wherein the interface to receive personal data is configured to access at least one of
storage media including electronic patient data, and
medical image transfer systems.

12. The system of claim 9, further comprising:
a determination unit and/or a notification unit to at least one of automatically determine, reserve and notify capacities of emergency and diagnostic equipment based on the determined possible injuries.

13. A vehicle comprising:
a sensor system to record incident data, the sensor system being equipped with an interface configured to communicate with the interface to recieve incident data of the system of claim 9.

14. A non-transitory computer program product including a computer program, directly loadable into a memory device of a computer device, including program sections for performing the method of claim 1 when the computer program is executed in the computer device.

15. A non-transitory computer-readable medium storing program sections for execution by a computer unit, the program sections being configured to perform the method of claim 1 when the program sections are executed by the computer unit.

16. The method of claim 2, wherein the incident data includes data belonging to a group including at least one of
incident description
speed,
direction of movement,
relative positions,
relative orientation of at least one of a person or of persons and objects involved,
description of objects involved,
at least one of forces and accelerations acting on persons,
partial accelerations of parts of the body of persons,
depth of penetration of objects into persons, and
physical effect of a plurality of persons subjected to the incident involving force or violence on at least one of the persons.

17. The method as claimed of claim 5, wherein
the modeling process takes into account interaction of the persons involved in the incident involving physical force or violence.

18. The method of claim 5, wherein the automatically generating of the estimation data for potential injury states via the modeling process includes at least one of a simulation and a dictionary-based search.

19. The method of claim 17, wherein the automatically generating of the estimation data for potential injury states via the modeling process includes at least one of a simulation and a dictionary-based search.

20. The system of claim 10, wherein the interface to receive incident data is configured to access a computing system via the Internet of Things (IoT), a computer cloud, wireless networks or internet protocols.

21. The system of claim 11, wherein the interface to receive personal data is configured at least one of
to access storage media including electronic patient data conforming to HL7 standards, and
to access medical image transfer systems conforming to DICOM standards.

22. The system of claim 10, wherein the interface to receive personal data is configured to access at least one of
storage media including electronic patient data, and
medical image transfer systems.

23. The system of claim 22, wherein the interface to receive personal data is configured at least one of
to access storage media including electronic patient data conforming to HL7 standards, and
to access medical image transfer systems conforming to DICOM standards.

24. A vehicle comprising:
a sensor system to record incident data, the sensor system being equipped with an interface configured to communicate with the interface to recieve incident data of the system of claim 11.

25. A vehicle comprising:
a sensor system to record incident data, the sensor system being equipped with an interface configured to communicate with the interface to recieve incident data of the system of claim 12.
